(19) United States
(12) Patent Application Publication
(10) Pub. No.: US 2011/0033388 A1
(43) Pub. Date: Feb. 10, 2011

(54) IMAGEABLE RODENT MODEL OF ASTHMA

(76) Inventors: Akihiro Hasegawa, San Diego, CA (US); Toshinori Nakayama, San Diego, CA (US); Meng Yang, San Diego, CA (US)

Correspondence Address: MORRISON & FOERSTER LLP, 12531 HIGH BLUFF DRIVE, SUITE 100, SAN DIEGO, CA, 92130-2040, US

(21) Appl. No.: 12/681,348

(22) PCT Filed: Oct. 1, 2008
(86) PCT No.: PCT/US08/78506

§ 371 Date: Oct. 28, 2010

Related U.S. Application Data
(60) Provisional application No. 60/976,749, filed on Oct. 1, 2007.

(51) Int. Cl. A61K 49/00; A01K 67/00

(52) U.S. Cl. 424/9.2; 800/9

(57) ABSTRACT

An imageable rodent model for asthma is described. The invention provides a rodent model for asthma wherein a rodent is provided with fluorescently labeled lymphocytes sensitized to an allergen which can be monitored after inducing an asthmatic response by the allergen. Methods to monitor trafficking of the fluorescently labeled cells in the rodent model for asthma are provided. Methods to determine the effectiveness of candidate drugs that regulate asthmatic responses using the rodent asthma model are also provided.

### RELATED APPLICATION

This application claims the benefit of priority under 35 U.S.C §119(e) from U.S. Provisional Patent Application No. 60/976,749, filed Oct. 1, 2007, which is hereby incorporated by reference in its entirety.

### TECHNICAL FIELD

The invention relates to a rodent model for asthma. More particularly, it concerns a rodent model for asthma which has fluorescently labeled cells whose trafficking can be monitored after the inducement of an asthmatic response. Methods to determine the effectiveness of candidate drugs that regulate asthmatic responses using the rodent asthma model are also provided.

### BACKGROUND ART

Asthma is an immunological disease characterized by the Th2-driven inflammation in the airways. Inflammation in the peribronchial space, with increased production of airway mucus, and airway hyperreactivity (AHR), are cardinal features of asthma.

Murine models of asthma have been widely used to study the diverse cellular events following an asthmatic response. Ovalbumin (OVA) challenge models of asthma offer many opportunities for increasing our understanding of the pathogenetic mechanisms underlying this disease, as well as for identifying novel therapeutic targets. (Kumar et al., Curr Drug Targets 2008; 9:485-94.) There is no single “classical” model, because numerous alternatives exist with respect to the choice of mouse strain, method of sensitization, route and duration of challenge, and approach to assessing the host response. The review of “classical” OVA challenge model of asthma in mice by Kumar et al. summarizes a spectrum of OVA-challenge mouse models of asthma, based on the choice of mouse strain, route, dose and duration of challenge, as well as method of sensitization. Recently, various mouse models of allergy and allergic asthma by clinically relevant allergens have been developed. (Fuchs & Braun, Curr Drug Targets 2008; 9:495-502.)

Antigen-induced mouse models of pulmonary allergic disease have proved particularly informative in the genetic dissection of inflammatory pathways in the lung. Kung et al. developed a method for inducing severe pulmonary eosinophilia in the mouse and also studied the numbers of eosinophils in blood and bone marrow and the response to corticosteroid treatment. (Kung et al., Int Arch Allergy Immunol. 1994; 105:83-90.) Animals were sensitized with alum-precipitated OVA and challenged with aerosolized OVA 12 days later when serum IgE levels were significantly elevated. Four to eight hours after challenge there were moderate increases in the number of eosinophils in the bone marrow and peripheral blood, but only a few eosinophils were observed in the lung tissue and in bronchioalveolar lavage (BAL) fluid. Twenty-four hours after challenge, there was a marked reduction of eosinophils in bone marrow, while the number of eosinophils peaked in the perivascular and peribronchial regions of the lung. Forty-eight hours after challenge, the highest number of eosinophils was found in the BAL fluid, making up>80% of all cells in that compartment. The high levels of eosinophils in the lung tissue and BAL fluid lasted for 2-3 days and was followed by a more moderate but persistent eosinophilia for another 10 days. Nonsensitized animals showed no significant changes in the number of eosinophils in BAL fluid, lungs, blood or bone marrow. Histopathological evaluation also revealed epithelial damage, excessive mucus in the lumen and edema in the submucosa of the airways.

Pauwels et al. developed a murine in vivo model of allergic airway inflammation characterized by the presence of IgE antibodies to an inhaled antigen, peribronchial infiltrates with an increased number of eosinophils, and increased airway responsiveness to nonantigenic bronchoconstrictor stimuli. (Pauwels et al., Am. J. Respir. Crit. Care Med. 1997; 156:S78-S81.) The C57 Black 6 (C57Bl/6) mice were actively sensitized on Day 0 by intraperitoneal injection of 10 μg of OVA adsorbed to 1 mg of alum and from Day 14 to 21 exposed daily to aerosolized OVA over a 30-min period. On Day 22, airway inflammation, characterized by the presence of peribronchial and peribronchiolar mixed cellular infiltrates and consisting mainly of mononuclear cells and eosinophils, could be demonstrated. This was reflected by an increase in the number of eosinophils in BAL fluid recovered from these animals. Furthermore, this inflammatory response was accompanied by an increase in airway responsiveness to carbachol. Ovalbumin-specific IgE antibodies could be demonstrated in the serum of the sensitized and exposed animals.

An in vivo murine model of antigen-induced airway hyperreactivity and inflammation was developed to investigate the possibility, suggested by a wealth of descriptive human data, that alterations in immunoregulation are important in the genesis of airway hyperreactivity. (Gavett et al., Am J Respir Cell Mol Biol. 1994; 10:587-93.) A/J mice developed airway hyperreactivity and markedly increased numbers of pulmonary inflammatory cells following intraperitoneal sensitization and intratracheal challenge with sheep red blood cells. Notably, eosinophils were a prominent component of the inflammatory infiltrate. The dependence of these phenomena, both pathologic and functional, on CD4⁺ T lymphocytes was investigated by in vivo depletion of CD4⁺ cells using the anti-CD4 mAb GK1.5. When administered before antigen challenge, GK1.5 completely prevented both airway hyperreactivity and the infiltration of eosinophils. This model provides the first direct demonstration of the dependence of airway hyperreactivity upon CD4⁺ T lymphocytes, and the results are consistent with the possibility that eosinophils are effectors of this response.

U.S. patent application Ser. No. 11/568,896 (Publication No. US 2008-0172751) describes a mouse model of COPD and Th1 asthma induced by OVA and double stranded RNA (dsRNA). BALB/c mice (Jackson Lab, USA) were sensitized by administrating synthesized dsRNA polyinosinic-polycytidylic acid (PolyIC, Sigma, USA) and OVA intranasally, singly or together, four times. Ten days later, the mice were challenged with the intranasal administration of OVA to induce asthma. The resultant mice were named Th1 asthma mice. The negative control mice were administered only with phosphate buffered saline (PBS).

U.S. Pat. No. 6,215,040 describes a transgenic mouse that constitutively expressed IL-5 in lung epithelium resulting in a dramatic accumulation of peribronchial eosinophils and striking pathological changes including expansion of bronchus-associated lymphoid tissue (BALT), goblet cell hyperplasia, epithelial hypertrophy and focal collagen deposits. Surprisingly, these changes were not accompanied by a prominent eosinophil infiltration into the airway lumen. Thus, lung-specific expression of IL-5 alone (i.e., in the absence of antigen-induced pulmonary inflammation) can induce many of the pathologic changes associated with allergic respiratory disease. Moreover, these mice displayed AHR in response to methacholine challenge. Thus, AHR can occur without extensive infiltration of the airway lumen by eosinophils.

Two novel models of allergic asthma have been developed in mice receiving the same allergen sensitization, but with acute or chronic allergen exposures, the latter to mimic the human situation more closely. (Fernandez-Rodriguez et al., Int Immunopharmacol. 2008; 8:756-63.) OVA-sensitised mice were challenged by OVA inhalation twice on the same day for the acute model, and 18 times over a period of 6 weeks for the chronic model. Lung function was monitored in conscious, unrestrained mice immediately after the last challenge for up to 12 h. Airway responsiveness to inhaled methacholine and serum antibody levels were determined 24 h after challenge. Bronchioalveolar inflammatory cell recruitment was determined at 2 or 24 h. Acute and chronically treated mice had similar early and late asthmatic responses peaking at 2 h and 7-8 h, respectively. IgE and IgG antibody levels, compared with naïve mice, and eosinophil infiltration, compared with naïve and saline challenge, were elevated. Airway hyperresponsiveness to methacholine was observed 24 h after challenge in both models. The acute model had higher levels of eosinophilia, whereas the chronic model showed hyperresponsiveness to lower doses of methacholine and had higher levels of total IgE and ovalbumin-specific IgG antibodies. Both novel murine models of allergic asthma bear a close resemblance to human asthma, each offering particular advantages for studying the mechanisms underlying asthma and for evaluating existing and novel therapeutic agents.

Murine models of asthma have proved to be extremely useful for examination of the basic mechanisms of allergic inflammation and the underlying immunologic response. Many investigations revealed crucial roles for CD4⁺ type-2 helper T (Th2) cells and eosinophils in asthma. CD4⁺ Th2 cells, which are thought to be present in the airways of all patients with asthma, secrete key cytokines, such as IL-4 and IL-13, as well as IL-5 and IL-9. Conventional CD4⁺ T cells recognize exogenous antigens and initiate allergic inflammation in the lungs and, in mouse models of asthma, elimination of CD4⁺ cells abrogates the development of AHR. Similarly crucial to the pathogenesis of asthmatic inflammation are the so-called Th2 cytokines, interleukin (IL)-5 and IL-13 in particular. (See, e.g., Nakajima et al., Am. Rev. Respir. Dis. 1992; 146:374-377; Wills-Karp et al., Science 1998; 183:195-201.)

Although Th2-driven immune responses are vitally important in the development of asthma, in itself a Th2 response is not sufficient to induce asthma. A better understanding of the role of regulatory cells in asthma may lead to the identification of novel therapeutic targets. In the majority of clinical studies, pulmonary eosinophilia has been recognized as a predominant feature of the inflammatory infiltrate, which often correlates with disease severity. Recently, there has been increasing interest in the involvement of eosinophils in the pathogenesis of asthma. (Weller, P. F., Curr. Opin. Immunol. 1994; 6:85-90.) A spectrum of CD4⁺ T cells, including Th3 cells, T_{R} cells, CD4⁺CD25⁺ cells and NKT cells play a critical role in regulating this disease. Using natural killer T (NKT) cell-deficient mice, Akbari et al. show that allergen-induced airway hyperreactivity (AHR), a cardinal feature of asthma, does not develop in the absence of Vα14i NKT Cells. (Akbari et al., Nat. Med. 2003; 9:582-588.) Thus, pulmonary Vα14i NKT cells crucially regulate the development of asthma and Th2-biased respiratory immunity against nominal exogenous antigens. Miyahara et al. suggest an important role for effector CD8⁺ T cells in the development of AHR and airway inflammation, which may be associated with their Tc2-type cytokine production and their capacity to migrate into the lung. (Miyahara et al., Nat. Med. 2004; 10:865-869.) Wyss et al. described a model of ovalbumin-induced adenosine hyper-reactivity developed in BALB/c mice in which they determined that the adenosine-induced hyper-reactivity in mice was mast cell dependent. (Wyss et al., Br J. Pharmacol. 2005; 145: 845-852.)

Despite the various animal models of asthma described in the prior art, there is scant information regarding the migration and dynamics of antigen-specific Th2 cells into the asthmatic lung. This is partly due to the difficulty encountered in monitoring the cell trafficking in the asthmatic lung, especially in vivo. A common approach is to identify total and differential cell counts in bronchioalveolar lavage (BAL) fluid. However, the increase in number and percentage of eosinophils in short-term high-level challenge models does not reflect what happens in patients. Assessment of inflammatory response in tissue sections is more reliable, but time consuming. Thus, an animal model for asthma which allows easy monitoring of the cell trafficking in the asthmatic lung in vivo is badly needed in the art.

Fluorescent proteins have been used as fluorescent labels for a number of years. The originally isolated protein emitted green wavelengths and came to be called green fluorescent protein (GFP). Because of this, green fluorescent protein became a generic label for such fluorescent proteins in general, although proteins of various colors including red fluorescent protein (RFP), blue fluorescent protein (BFP) and yellow fluorescent protein (YFP) among others have been prepared. The nature of these proteins is discussed in, for example, U.S. Pat. Nos. 6,232,523; 6,235,967; 6,235,968; and 6,251,384. These patents describe the use of fluorescent proteins of various colors to monitor tumor growth and metastasis in transgenic rodents which are convenient tumor models.

A dual-color fluorescence imaging model of tumor-host interaction based on an RFP-expressing tumor growing in GFP transgenic mice, enabling dual-color visualization of the tumor-stroma interaction including tumor angiogenesis and infiltration of lymphocytes in the tumor has been described. Transgenic mice expressing the GFP under the control of a chicken beta-actin promoter and cytomegalovirus enhancer were used as the host (Okabe et al., FEBS Lett 1997; 407:315-319). All of the tissues from this transgenic line fluoresce green under blue excitation light. RFP-expressing B16F0 (B16F0-RFP) mouse melanoma cells were transduced with the pLNCX₂-DsRed-2-RFP plasmid. The B16F0-RFP tumor and GFP-expressing host cells could be clearly imaged simultaneously. High-resolution dual-color images enabled resolution of the tumor cells and the host tissues down to the single cell level. Host cells including fibroblasts, tumor infiltrating lymphocytes, dendritic cells, blood vessels and capillaries that express GFP, could be readily distinguished from the RFP-expressing tumor cells. This dual-color fluorescence imaging system should facilitate studies for understanding tumor-host interaction during tumor growth and tumor angiogenesis. The dual-colored chimeric system also provides a powerful tool to analyze and isolate tumor infiltrating lymphocytes and other host stromal cells interacting with the tumor for therapeutic and diagnostic/analytic purposes. The above reference is incorporated herein by reference.

Recently, Yang et al. conducted whole-body optical imaging of GFP-expressing tumors and metastases (Yang et al., Proc. Natl. Acad. Sci. (USA) 2000; 97:1206-11). Yang et al. have imaged, in real time, fluorescent tumors growing and metastasizing in live mice. The whole-body optical imaging system is external and noninvasive. It affords unprecedented continuous visual monitoring of malignant growth and spread within intact animals. Yang et al. have established new human and rodent tumors that stably express very high levels of the Aequorea victoria GFP and transplanted these to appropriate animals. B 16F0-GFP mouse melanoma cells were injected into the tail vein or portal vein of 6-week-old C57BL/6 and nude mice. Whole-body optical images showed metastatic lesions in the brain, liver, and bone of B16F0-GFP that were used for real time, quantitative measurement of tumor growth in each of these organs. The AC3488-GFP human colon cancer was surgically implanted orthotopically into nude mice. Whole-body optical images showed, in real time, growth of the primary colon tumor and its metastatic lesions in the liver and skeleton. Imaging was with either a trans-illuminated epifluorescence microscope or a fluorescence light box and thermoelectrically cooled color charge-coupled device camera. The depth to which metastasis and micrometastasis could be imaged depended on their size. A 60-micrometer diameter tumor was detectable at a depth of 0.5 mm whereas a 1,800-micrometer tumor could be visualized at 2.2-mm depth. The simple, noninvasive, and highly selective imaging of growing tumors, made possible by strong GFP fluorescence, enables the detailed imaging of tumor growth and metastasis formation. This should facilitate studies of modulators of cancer growth including inhibition by potential chemotherapeutic agents. The whole-body external fluorescent optical imaging technology shown above is disclosed in U.S. Pat. No. 6,649,159.

### DISCLOSURE OF THE INVENTION

The present invention is directed to a rodent model for asthma with fluorescently labeled cells whose trafficking can be monitored after an asthmatic response has been induced. The model is a rodent that has been provided allergen-sensitized, fluorescently labeled lymphocytes which can be detected after inducing an asthmatic response to said allergen. In another aspect, the invention is directed to a method to monitor cell trafficking in the rodent asthma model. In yet another aspect, the invention is directed to methods to screen for anti-asthma drugs using the rodent model by looking for drugs that specifically inhibit the trafficking of the fluorescent cells responsible for the asthmatic response.

### BRIEF DESCRIPTIONS OF THE DRAWINGS

FIG. 1. Dual color visualization of GFP⁺ CD4⁺ T cell infiltration into the lung in OVA-induced allergic asthma.

FIG. 2. Dual color visualization of RFP⁺ CD4⁺ T cell infiltration into the lung in OVA-induced allergic asthma.

FIG. 3. Visualization of CD8⁺ T cell infiltration into the lung in OVA-induced allergic asthma.

FIG. 4. Time course and Dexamethasone inhibition of CD4⁺ T cell accumulation in the lung.

FIG. 5. Time course and Dexamethasone inhibition of CD4⁺ T cell accumulation in the lung by fluorescent imaging.

FIG. 6. Visualization of OT2-Th2 cell accumulation into the lung in OVA-induced allergic asthma.

FIG. 7. Histological and immunohistochemical analysis for the induction of inflammatory foci and GFP⁺ Th2 cell foci after allergen challenge.

### MODES OF CARRYING OUT THE INVENTION

The tools useful in the present invention are described in the publications, U.S. patents, and patent applications incorporated by reference above. Whole body imaging, the nature of fluorescent proteins useful in the invention, and methods to label entire animals have been described in these documents.

In order to avoid confusion, the simple term “fluorescent protein” will be used; in general, this is understood to refer to the fluorescent proteins which are produced by various organisms, such as Renilla and Aequorea as well as modified forms of these native fluorescent proteins which may fluoresce in various visible colors, such as red, yellow, and cobalt, which are exhibited by red fluorescent protein (RFP), yellow fluorescent protein (YFP) or cobalt fluorescent protein (CFP), respectively. In general, the terms “fluorescent protein” and “GFP” or “RFP” are used interchangeably.

The invention provides a rodent model for asthma wherein the rodent has been provided allergen-sensitized, fluorescently labeled lymphocytes which can be detected after inducing an asthmatic response to said allergen.

In a specific embodiment, the fluorescently labeled lymphocytes can be T lymphocytes. In another specific embodiment, the fluorescently labeled lymphocytes can be CD4⁺ T lymphocytes. In yet another specific embodiment, the fluorescently labeled lymphocytes can be Th2 cells.

Any of the protocols to induce asthmatic responses described in the above references can be used in this model. The rodent may be, for instance, a mouse or rat. Mouse strains suitable may be BALB/c, C57BL/6, B6D2F1/J, A/J, CBA/J, etc. Allergens for challenge and sensitization may be OVA, OVA peptide, sheep red blood cells, dsRNA, cockroach (rBla g2), house dust mite (rDer f1), house dust mite-extract, olive pollen (natural and recombinant Ole e1), Aspergillus fumigatus-extract, Timothy grass pollen (rPhl p5), birch pollen (rBet v1), rye grass pollen (Lol p1), olive pollen extract, Alternaria alternate-extract, Cladosporium herbarum-spores, Dermatophagoides pteronyssinus-extract, heat-coagulated hen's egg white, etc., or any combination thereof. The route of challenge may be inhalation, or intratracheal, intranasal, intraperitoneal, or subcutaneous injection, etc.

In a specific embodiment, the fluorescently labeled cells come from a donor mouse which expresses a fluorescent protein ubiquitously or in a subset of cells. The donor mouse may be sensitized prior to the collection of the fluorescently labeled cells to be introduced to the recipient mouse. Sensitization may be performed with either of the above listed allergens, or any combination thereof, with or without an adjuvant(s). Adjuvants used may be alum, HDM/CFA,¹ IFA,² PT,³ etc., or any combination thereof. The route of sensitization may be intraperitoneal, intranasal, intratracheal, or subcutaneous injection, etc. ¹ CFA=complete Freud's adjuvant² IFA=incomplete Freud's adjuvant³ PT=pertussis toxin

In another specific embodiment, the fluorescently labeled cells come from two donor animal which express two different fluorescent proteins. One donor is sensitized while the other donor is not sensitized and is used as control. The two distinctively labeled cell populations are introduced to the same recipient animal and their trafficking may be monitored simultaneously by dual-color fluorescence imaging described above.

This invention further provides a method to determine the effectiveness of candidate anti-asthma drugs by administering the substance to the rodent model for asthma followed by monitoring the inhibitory effects on cell trafficking. Administration of the candidate substance can be performed before, after, or simultaneously with the challenge on the animal to induce asthmatic responses. Dual-color fluorescent imaging may be used to monitor the effects of the candidate substance on sensitized and control cell populations in order to filter out false positives.

Cell trafficking may be monitored in tissue sections that have been excised, in living tissues ex vivo, or in living animals in vivo. For in vivo monitoring of living animals, either endoscopy or whole-body fluorescent imaging may be performed, which is described in more detail in the sections that follow.

The label used in the various aspects of the invention is a fluorescent protein. The native gene encoding the seminal protein in this class, GFP, has been cloned from the bioluminescent jellyfish Aequorea victoria (Morin et al., J. Cell Physiol. 1972; 77:313-318). The availability of the gene has made it possible to use GFP as a marker for gene expression. The original GFP itself is a 283 amino acid protein with a molecular weight of 27 kD. It requires no additional proteins from its native source nor does it require substrates or cofactors available only in its native source in order to fluoresce. (Prasher et al., Gene 1992; 111:229-233; Yang et al., Nature Biotechnol. 1996; 14:1252-1256; Cody et al., Biochemistry 1993; 32:1212-1218.) Mutants of the original GFP gene have been found useful to enhance expression and to modify excitation and fluorescence, so that “GFP” in various colors, including reds and blues has been obtained. GFP-S65T (wherein serine at 65 is replaced with threonine) is particularly useful in the present invention method and has a single excitation peak at 490 nm (Heim et al., Nature 1995; 373:663-664; U.S. Pat. No. 5,625,048.) Other mutants have also been disclosed by Delagrade et al., Biotechnology 1995; 13:151-154; Cormack et al., Gene 1996; 173:33-38; and Cramer et al., Nature Biotechnol. 1996; 14:315-319. Additional mutants are also disclosed in U.S. Pat. No. 5,625,048. By suitable modification, the spectrum of light emitted by the GFP can be altered. Thus, although the term “GFP” is often used in the present application, the proteins included within this definition are not necessarily green in appearance. Various forms of GFP exhibit colors other than green and these, too, are included within the definition of “GFP” and are useful in the methods and materials of the invention. In addition, it is noted that green fluorescent proteins falling within the definition of “GFP” herein have been isolated from other organisms, such as the sea pansy, Renilla reniformis. Any suitable and convenient form of GFP can be used to modify the infectious agents useful in the invention, both native and mutated forms.

The methods of the invention utilize fluorescently labeled cells, preferably of sufficient fluorescence intensity that the fluorescence can be seen in the subject without the necessity of any invasive technique. While whole body imaging is preferred because of the possibility of real-time observation, endoscopic techniques, for example, can also be employed or, if desired, tissues or organs excised for direct or histochemical observation.

Although endoscopy can be used as well as excision of individual tissues, it is particularly convenient to visualize the migration of cells in the intact animal through fluorescent imaging. This permits real-time observation and monitoring of cell trafficking on a continuous basis, in particular, in model systems, in evaluation of potential anti-asthma drugs and protocols. Thus, the inhibition of cell trafficking observed directly in test animals administered a candidate drug or protocol in comparison to controls which have not been administered the drug or protocol indicates the efficacy of the candidate and its potential as a treatment. In subjects being treated for asthma, the availability of fluorescent imaging permits those devising treatment protocols to be informed on a continuous basis of the advisability of modifying or not modifying the protocol.

Fluorescence imaging (See Yang, M., Proc. Natl. Acad. Sci. USA 2002; 99:3824-3829). A Leica fluorescence stereo microscope model LZ12 equipped with a mercury 50W lamp power supply is used for initial lower resolution imaging. For visualization of both GFP and RFP fluorescence simultaneously, excitation is produced through a D425/60 band pass filter and 470 DCXR dichroic mirror. Emitted fluorescence is collected through a long pass filter GG475 (Chroma Technology, Brattleboro, Vt.). Macroimaging is carried out in a light box (Lightools Research, Encinitas, Calif.). Fluorescence excitation of both GFP and RFP tumors is produced in the lightbox through an interference filter (440+/−20 nm) using slit fiber optics. Fluorescence is observed through a 520 nm long pass filter. Images from the microscope and light box are captured on a Hamamatsu C5810 3-chip cool color CCR camera (Hamamatsu Photonics Systems, Bridgewater, N.J.). Laser-based imaging is carried out with the Spectra Physics model 3941-M1BB dual photon laser, Photon Technology Intl. model GL-3300 nitrogen laser and the Photon Technology Intl. model GL-302 dye laser. Images are processed for contrast and brightness and analyzed with the use of Image Pro Plus 4.0 software (Media Cybernetics, Silver Springs, Md.). High resolution images of 1024×724 pixels are captured directly on an IBM PC or continuously through video output on a high resolution Sony VCR model SLV-R1000 (Sony Corp., Tokyo Japan).

Multiphoton confocal microscopy (Wang et al., Cancer Res. 2002; 62:6278-6288). The dual photon laser (Spectra-Physics model 3941-M1BB) is also used with the Radiance 2000 multiphoton system (Bio-Rad, Hercules, Calif.) at 960 nm, the optimal wavelength for GFP fluorescence. The images are collected using Bio-Rad's Lasersharp 2000 software. Excitation is confined only to the optical section being observed. No excitation of the fluorophore will occur at 960 nm wavelength not in the plane of focus. The Millenia, Tsunami Ti:Sapphire laser, an accessory for the Spectra Physics model 3941-M1BB dual photon laser, has long wavelength optics (beyond 1,000 nm) for RFP multiphoton imaging. Images are processed with Image Pro Plus 4.0 software.

Spectral resolution. Spectral imaging, is the generation of images containing a high-resolution optical spectrum at every pixel, to “unmix” the RFP signal from that of the GFP-labeled cells. The standard GFP-mouse imaging system (long-pass emission filter) is modified by replacing the usual color camera with the cooled monochrome camera (Roper Scientific CCD thermo-cooled digital camera) and a liquid crystal tunable filter (CRI, Inc., Woburn, Mass.) positioned in front of a conventional macro-lens. Typically, a series of images is taken every 10 nm from 500 to 650 nm and assembled automatically in memory into a spectral “stack.” Using pre-defined GFP or RFP and autofluorescence spectra, the image can be resolved into different images using a linear combination chemometrics-based algorithm that generates images containing only the autofluorescence signals or only the GFP or RFP signals, now visible against essentially a black background. Using spectral autofluorescence subtraction, sensitivity is enhanced due to improvements in signal to noise ratio. The advantages provided by the GFP- or RFP-labeled cells, which allow noninvasive, and highly selective imaging, are further enhanced by using wavelength-selective imaging techniques and analysis to image cell trafficking on deep organs such as the lung (personal communication, Richard Levenson, CRI, Inc., Woburn, Mass.).

Depth of imaging. External visualization of single cells or microscopic colonies of cells on internal organs is one goal of this application. Imaging of this power requires reducing scatter of excitation and emission light. Multiphoton and single photon lasers will be used for deeper penetration in the living animal. Confocal microscopy will also be used in conjunction with the multiphoton laser. The relatively high wave length of the excitation light, about 470 nm (960 nm for GFP dual photon and about 1,220 nm for RFP dual photon), will not damage tissue. The multiphoton confocal system will highly limit the irradiation area further protecting the host tissues. Skin-flaps also greatly reduce scatter which we have already shown to enable external single-cell imaging. Use of the long wave length Ds-Red-2-RFP also reduces scatter.

The following examples are offered to illustrate but not to limit the invention.

### A. Methods

Mice. C57BL/6 were purchased from Charles River Laboratories. C57BL/6-Tg(CAG-EGFP)C14-Y01-FM131Osb (GFP Tg, C57BL/6 background) mice expressing an enhanced GFP in the whole body (Okabe et al., FEBS Lett. 1997; 407:313-319) were provided by Dr. Okabe (Osaka University, Japan). OVA-specific TCRαβ transgenic (OT2 Tg) mice were maintained under specific-pathogen-free conditions. All animal care was carried out in accordance with guidelines of Chiba University and AntiCancer, Inc.

In vitro Th2 cell differentiation cultures. GFP×OT2 Tg CD44^{low}CD4⁺ T cells (2×10⁵) purified by cell sorting were stimulated with antigenic OVA peptide (Loh 15, 1μM) and irradiated (3000 rad) C57BL/6 antigen presenting cells (1×10⁶) in the presence of exogenous IL-4 as described previously (Hasegawa et al., J. Immunol. 2006; 176:2546-2554).

OVA-sensitization, cell transfer and OVA-inhalation. GFP or RFP Tg mice were immunized intraperitoneally with 250 μg OVA (chicken egg albumin from Sigma) in 4 mg aluminum hydroxide gel (alum) on day 0 and 7. Splenic CD4⁺ T cells from OVA-sensitized GFP or RFP Tg mice were isolated by magnetic negative selection using a CD4⁺ T cell isolation kit (Miltenyi Biotec) on day 14, yielding a purity of >98%. These cells (2×10⁷ cells) or OVA-specific Th2 cells (5×10⁶ cells) were transferred intravenously through the tail vain to 8-wk-old C57BL/6 recipient mice. One or two days later, the recipient mice inhaled aerosolized OVA in saline (10 mg/ml) for 30 min using a supersonic nebulizer (NE-U07, Omron Co. Japan).

Lung histology and immunohistochemistry. Mice were sacrificed by CO₂ asphyxiation at indicated time after the OVA inhalation, and the lungs were infused with 10% (v/v) formalin in PBS or 4% (v/v) paraformaldehyde for fixation. The lung samples were sectioned, stained with H&E reagents, and examined for pathological changes under a light microscope at ×50 or ×200. Lung specimens were embedded in Tissue-Tek OCT compound, frozen in liquid nitrogen, and cut by a cryostat into 6-μm-thick sections. The endogenous peroxidase activity as well as nonspecific protein binding was sequentially blocked using 0.6% hydrogen peroxide and Biotin-Blocking System reagent (DAKOCytomation), respectively. The sections were incubated with hamster anti-GFP mAb (Serotec) at 10 μg/ml overnight at 4° C. and were then washed in TBST. Bound Ab was detected by sequential incubation with biotinylated rabbit anti-hamster IgG and streptavidin-HRP followed by 3,3-diaminobenzidine (DAKOCytomation). Slides were then washed in water and counterstained with hematoxylin.

Visualization of cell trafficking in the lung. Mice were killed by CO₂ asphyxiation at various times after OVA inhalation. Lungs were removed, and GFP⁺ and RFP⁺ cells on the surface of the lung were monitored using the OV100 Olympus Whole Mouse Imaging System.

Laser scanning microscopy for in vivo movie. Mice were anesthetized and tracheostomized Lungs were exposed microsurgically. A right bronchus was clipped to stop the movement by ventilation. Left lung was mechanically ventilated to keep alive. The clipped right lung was monitored by a laser scanning microscope, IV100 (Olympus Corp.). A 488-nm argon laser was used. To create an in vivo movie, images were recorded with 5 sec intervals for 40 min. Focus area is prescribed by where more than 50% of 2D-area is occupied by the GFP⁺ cells. Motive cells in the lung were prescribed by which migrate or elongate more than 50% of the diameter.

Statistical analysis. Experimental data were expressed as the mean with standard deviations. The significance between two groups was determined by two-tailed Student's t test.

### B. Results

### Example 1

GFP Tg mice were sensitized with OVA-alum on days 0 and 7.

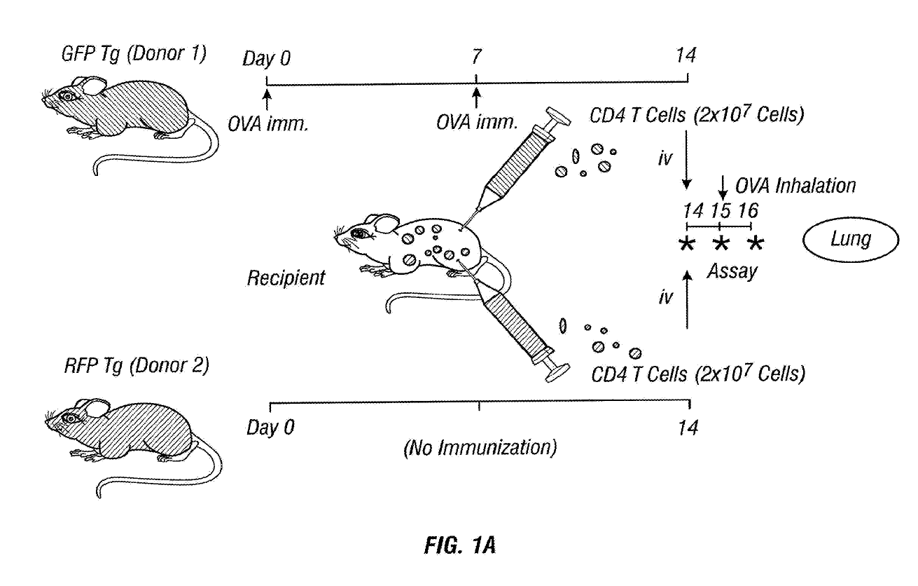

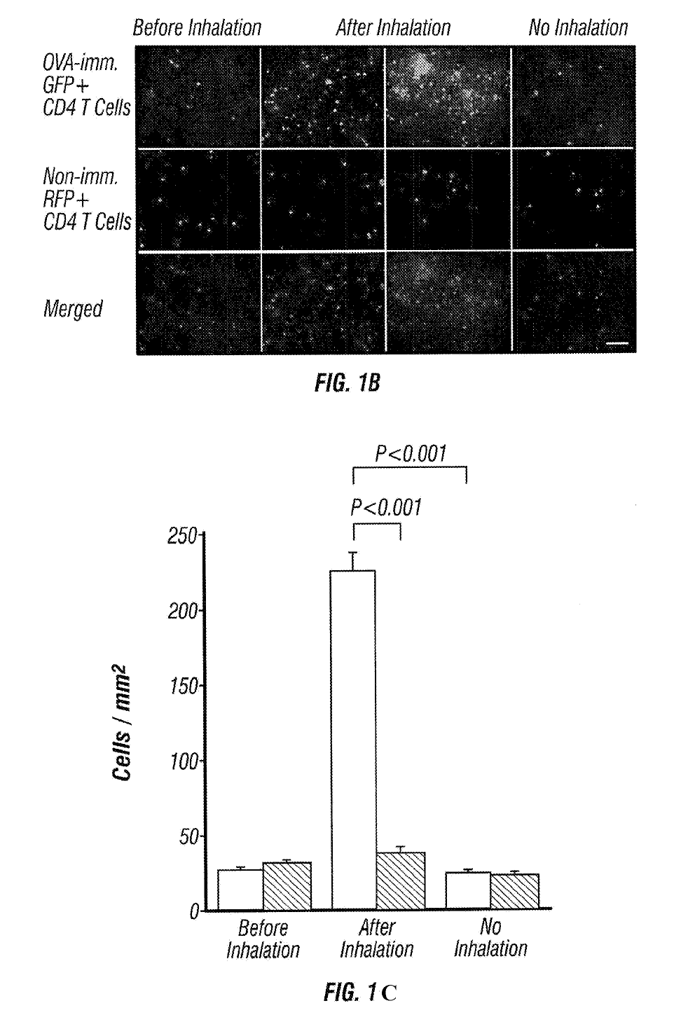

Splenic CD4⁺ T cells from OVA-sensitized GFP Tg and non-sensitized RFP Tg mice were purified and injected into normal C57BL/6 mice on day 14. The recipient mice were exposed to aerosolized OVA allergen challenge by airway administration on day 15. On day 16, GFP⁺ and RFP⁺ CD4⁺ T cells on the surface of the lung were monitored by OV100 microscopy (FIG. 1a).

Immediately after injection, large numbers of transferred cells were accumulated in the lung capillaries (FIG. 1b). Similar numbers of GFP⁺ and RFP⁺ cells were detected. One day after cell transfer, there was no significant number of GFP⁺ and RFP⁺ cells remaining in the lung. Twenty four hours after OVA inhalation, however, the number of GFP⁺ CD4⁺ T cells from OVA-sensitized mice increased significantly and some of them formed foci that look like clusters of CD4⁺ T cells. On the other hand, the number of RFP⁺ CD4⁺ T cells from non-sensitized mice did not increase (FIGS. 1b,c). These results indicate that CD4⁺ T cell migration into the lung after OVA inhalation is dependent on priming with OVA.

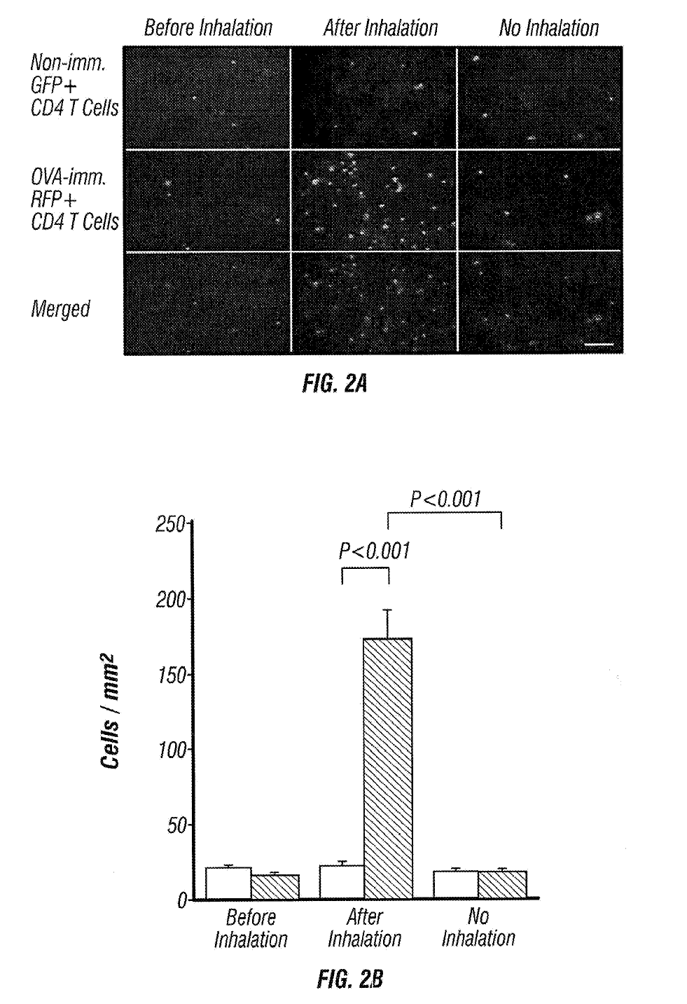

If the sensitized CD4⁺ T cells were labeled with RFP, only these accumulated in the lung after OVA inhalation, but not non-sensitized GFP⁺ CD4⁺ T cells (FIGS. 2a,b). These results indicate that the difference of accumulation is not fluorescence protein dependent.

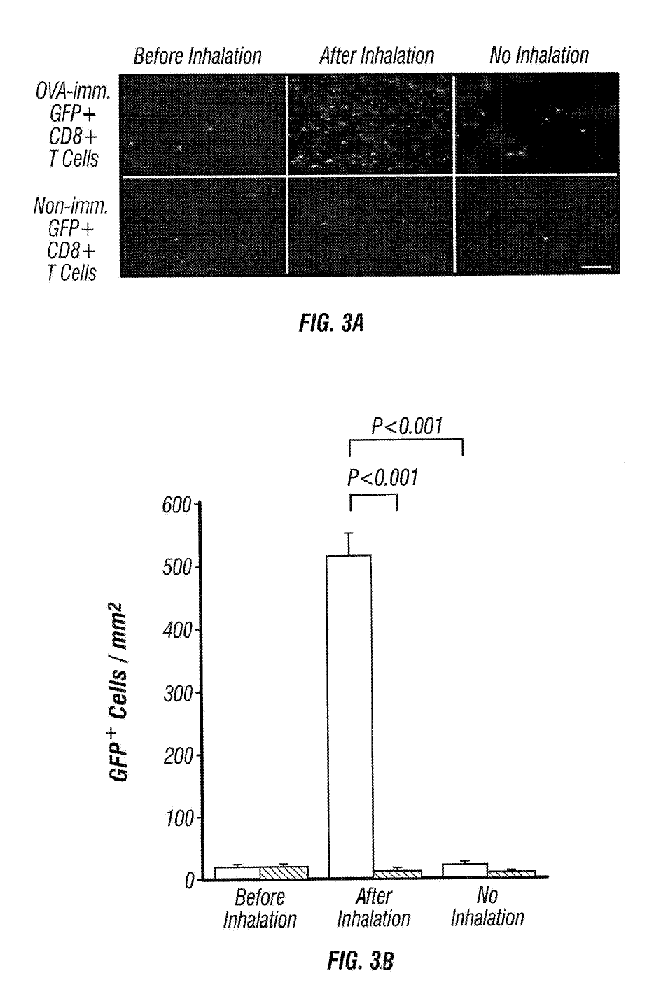

When splenic CD8⁺ T cells from OVA-sensitized GFP⁺ Tg mice were purified and injected into recipient mice, they also accumulated in the lung after OVA inhalation (FIG. 3).

### Example 2

The time course of CD4⁺ T cell accumulation in the lung after OVA inhalation was examined.

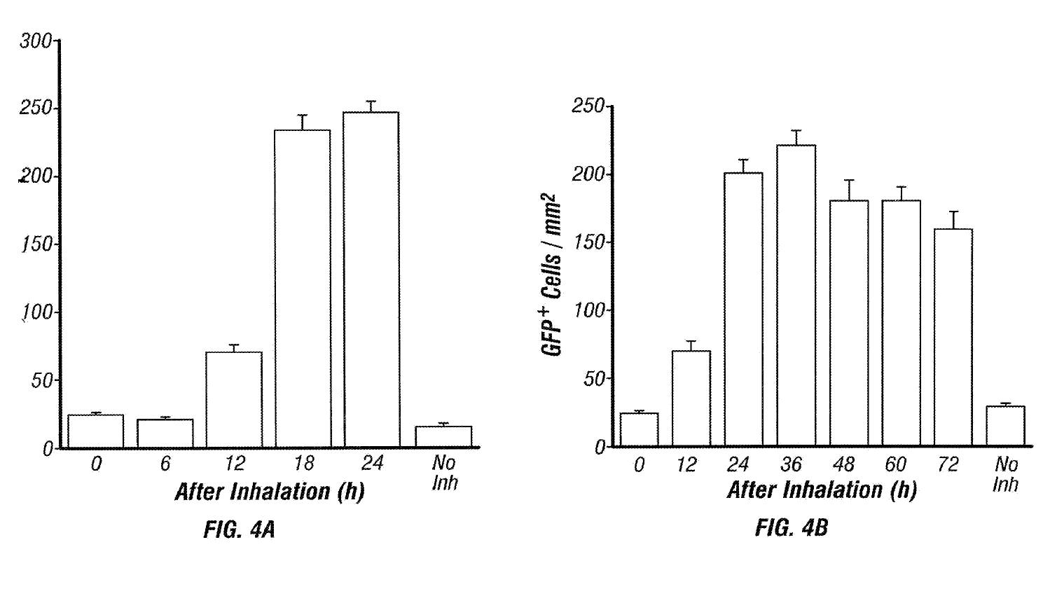

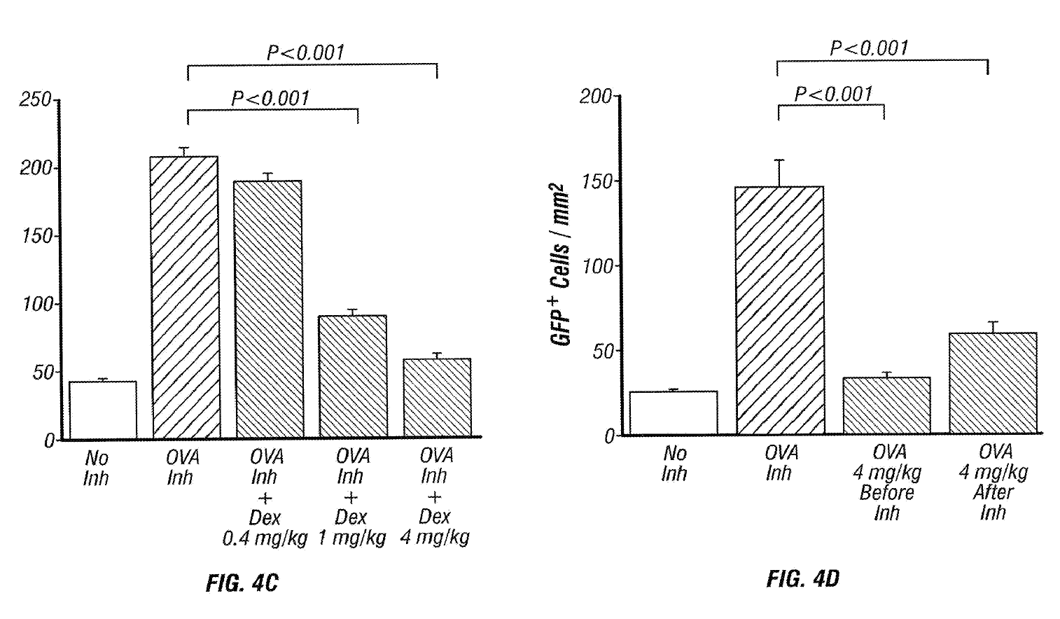

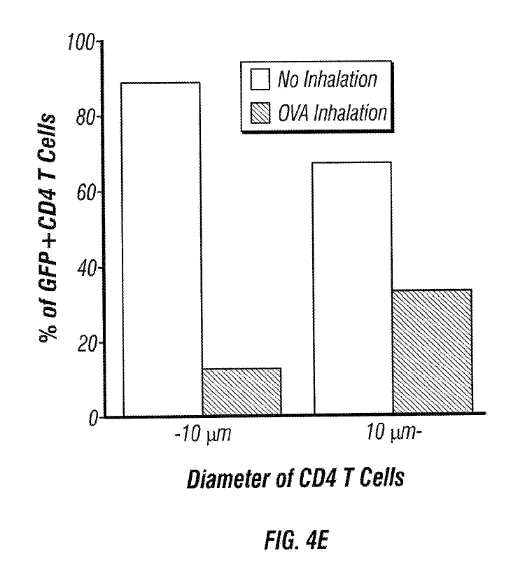

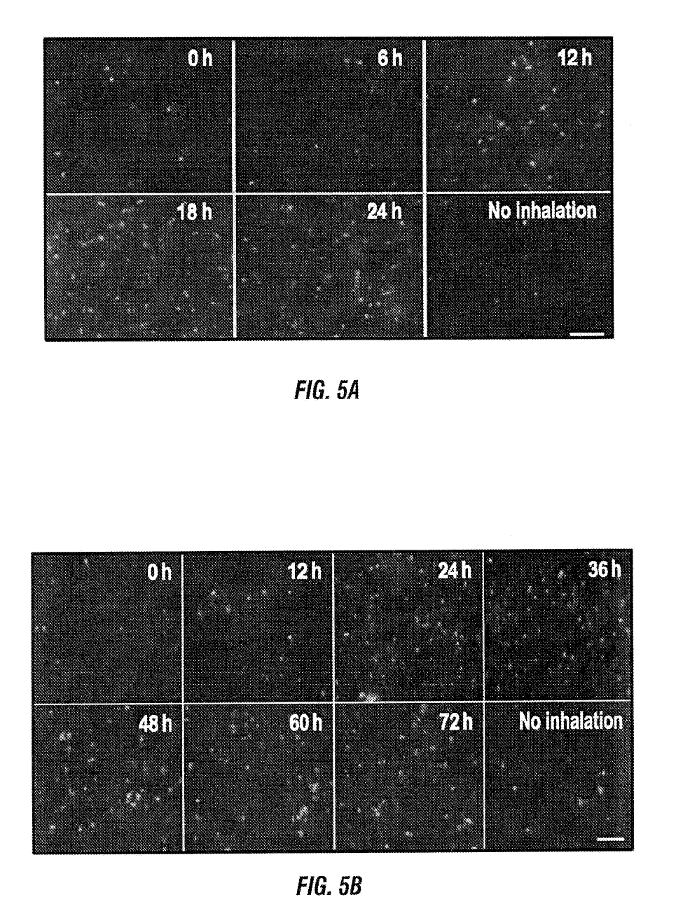

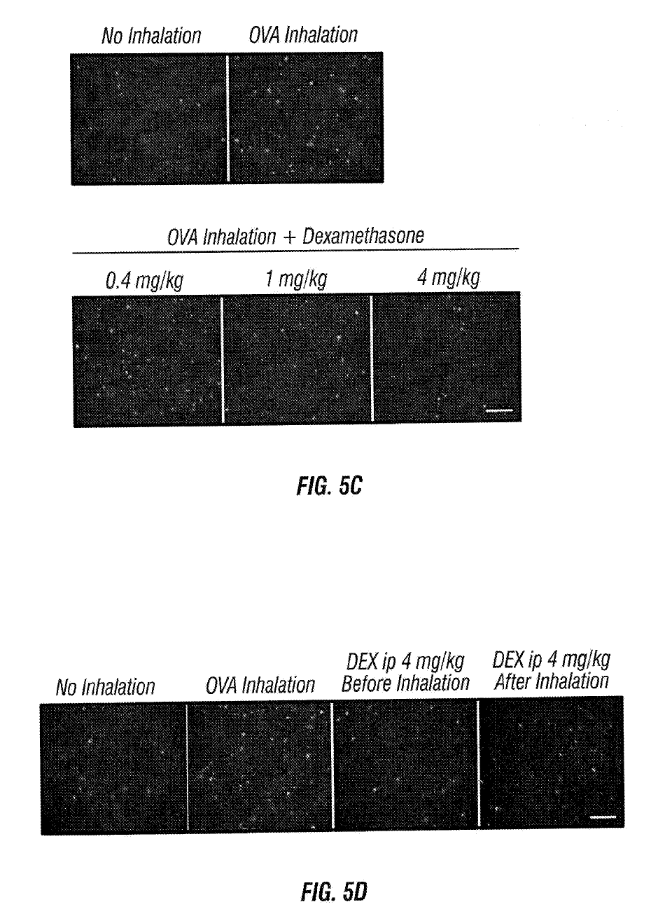

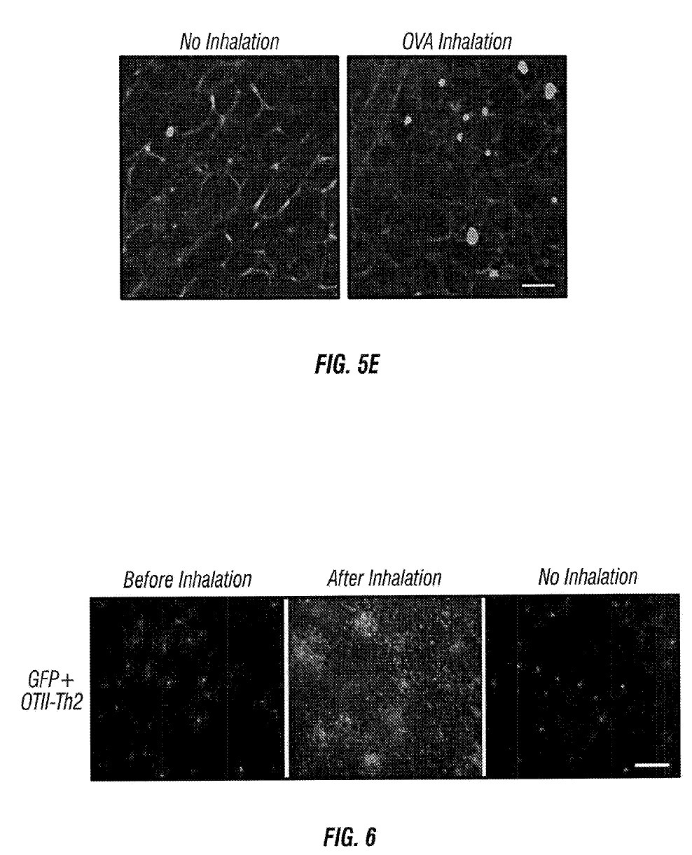

Splenic CD4⁺ T cells from OVA-sensitized GFP Tg mice were injected into recipient C57BL/6 mice, and the recipient mice were exposed to an allergen challenge as described in Example 1. GFP⁺ CD4⁺ T cells on the surface of the lung were monitored at 24 hours (FIGS. 4a and 5a) and 72 hours (FIGS. 4b and 5b) after OVA inhalation by OV100 microscopy. Migration of GFP⁺ CD4⁺ T cells into the lung was first detected at 12 hours after OVA inhalation, and the maximum number of CD4⁺ T cells was detected at 18 to 36 hours after OVA inhalation. While eosinophil infiltration is characteristic in allergic airway inflammation, these results indicate that CD4⁺ T cell accumulation in the lung after the allergen challenge occurs prior to infiltration of eosinophils, and sustains to at least 72 hours after the allergen challenge.

This imageable model proves useful to monitor the migration of inflammatory lymphocytes in the asthmatic lung. As a test for the model's utility for determining the effectiveness of an anti-allergy drug, Dexamethasone, a potent drug which attenuates allergic reactions, was administered to the recipient mice before the allergen challenge.

Three different doses (0.4, 1, and 4 mg/kg body weight) of Dexamethasone were injected intraperitoneally into the recipient mice 1 hour before the OVA challenge. Twenty-four hours after OVA inhalation, GFP⁺ CD4⁺ T cells were monitored by OV100 microscopy. A significant decrease in the CD4⁺ T cell infiltration to the lung as compared to controls occurred in a dose-dependent fashion (FIGS. 4c and 5c). These results suggest that Dexamethasone inhibits CD4⁺ T cell accumulation in the lung after an allergen exposure.

Further experiments were performed to test the time frame of Dexmethasone's anti-allergy effect. Dexamethasone (4 mg/kg) was injected intraperitoneally 1 hour before or 1 day after OVA inhalation, and GFP⁺ CD4⁺ T cells were monitored 48 hours after OVA inhalation. The numbers of infiltrating of GFP⁺ CD4⁺ T cells were significantly decreased in both cases (FIGS. 4d and 5d). These results indicate that Dexamethasone inhibits the infiltration of CD4⁺ T cells even if administered after the airway inflammation.

Next, we monitored the morphological changes of GFP⁺ CD4⁺ T cells in the lung after OVA inhalation by IV100 microscopy. Both autofluorescent endothelial cells and GFP⁺ CD4⁺ T cells in the lung were visualized (FIG. 5e). The mean diameter of CD4⁺ T cells in the challenged lung was significantly larger than the control (FIGS. 4e and 5e). These results indicate that the infiltrating CD4⁺ T cells are activated.

### Example 3

To investigate the dynamics of antigen-specific Th2 cells in the asthmatic lung, OVA-specific Th2 cells were induced in vitro from naive CD4⁺ T cells from GFP Tg×OT2 Tg mice.

First, we confirmed the accumulation of GFP⁺ OVA-specific Th2 cells in the lung after an allergen challenge. GFP⁺ OT2-Th2 cells accumulated in the lung after OVA inhalation more efficiently than CD4⁺ T cells from OVA-primed mice (FIG. 6). The number of foci with OT2-Th2 cells was much more than that with OVA-primed CD4⁺ T cells (data not shown). These results indicate that OT2-Th2 cells induced in vitro accumulate in the lung more efficiently after antigen inhalation.

Next, we performed time course analysis of OT2-Th2 cell accumulation after OVA inhalation. OT2-Th2 cell formed small foci 6 hours after OVA inhalation. The number and size of the foci increased 12 hours after OVA inhalation. GFP⁺ cell number in non-focus area also increased but not significantly until 12 hours after OVA inhalation. The number of foci further increased 18 h after OVA inhalation, and GFP⁺ cell number in non-focus area increased significantly between 12 and 18 hours after OVA inhalation. Eighteen hours after OVA inhalation or later, the border of foci became unclear and foci began to merge.

Next, we investigated the dynamics of OT2-Th2 cell infiltration in the lung after antigen exposure.

At the stable stage before OVA inhalation, no focus was observed in the lung and only 10% of OT2-Th2 cells in the lung were motive (Table 1). In vivo movies captured by laser scanning microscope IV100 showed that the number of circulating OT2-Th2 cells was 14.7±1.5 cells/mm²/30 min (Table 1). The number of circulating cells accumulated in the lung was 7.0±1.5 cells/mm²/30 min and the number of circulating cells exiting from the lung was 7.0±1.0 cells/mm²/30 min (Table 1). These results indicate that allergen-specific effecter T cells are circulating in the body and repeatedly entering into, accumulating in, and exiting from the lung. The number of cells entering into the lung and that exiting are even, and the number of effecter T cells in the lung is kept constant.

Six hours after OVA inhalation, small foci were observed. Compared with stable stage, in vivo movies showed that circulating OT2-Th2 cells increased to 33.3±3.1 cells/mm²/30 min, and accumulating cells increased to 14.7±1.5 cells/mm²/30 min (Table 1). The percentage of motive cells increased from 10% to 30.5%. On the other hand, OT2-Th2 cells exiting from the lung remained the same as stable stage. These observations indicate that allergen-induced migration and accumulation of OT2-Th2 cells into the lung were up-regulated by 6 hours after OVA inhalation.

Twelve hours after OVA inhalation, in vivo movies showed that larger foci were observed, and circulating OT2-Th2 cells into the lung further increased to 44.7±4.5 cells/mm²/30 min (Table 1). OT2-Th2 cells accumulation in the lung further increased to 24.3±2.5 cells/mm²/30 min, but OT2-Th2 cells exiting from the lung did not change significantly compared with stable stage (Table 1). Ninety percent of OT2-Th2 cells accumulating in the lung were motive. These results indicate that allergen-induced migration and accumulation of OT2-Th2 cells into the lung were highly up-regulated at 12 hours after OVA inhalation.

In the early stage of accumulation in the lung between 6 and 12 h after OVA inhalation, migrating OT2-Th2 cells form foci dominantly. High motility of accumulating OT2-Th2 cells in the lung suggests that most of them were activated.

Twenty-one hours after OVA inhalation, in vivo movies showed that OT2-Th2 cell number in non-focus area increased significantly compared with that of 12 h after OVA inhalation (868.7±296.5 vs.226.0±25.1 cells/mm²/30 min). Circulating OT2-Th2 cells into the lung significantly decreased compared with that of 12 h after OVA inhalation, from 44.7±4.5 to 2.7±0.6 cells/mm²/30 min (Table 1). OT2-Th2 cell accumulation in the lung and exiting from the lung also significantly decreased 21 h after OVA inhalation. More than 95% of accumulating cells were motive. These results indicate that allergen-induced migration and accumulation of OT2-Th2 cells into the lung was down-regulated by 21 h after OVA inhalation.

In the late stages of accumulation in the lung between 12 and 21 h after OVA inhalation, OT2-Th2 cells accumulated in the whole area of the lung in addition to the focus areas.

TABLE 1
OT2-Th2 Cell Accumulation in Non-Focus Area of the Lung.

| | After OVA Inhalation | | | |
|---|---|---|---|---|
| | 0 h | 6 h | 12 h | 21 h |
| Cells in Non-Focus Area | 57.7 ± 4.5 | 134.3 ± 16.9 | 226.0 ± 25.1 | 868.7 ± 296.5 |
| (cells/mm²) | | | | |
| Circulating Cells | 14.7 ± 1.5 | 33.3 ± 3.1 | 44.7 ± 4.5 | 2.7 ± 0.6 |
| (cells/mm²/30 min) | | | | |
| Accumulating Cells | 7.0 ± 1.5 | 14.7 ± 1.5 | 24.3 ± 2.5 | 1.3 ± 0.6 |
| (cells/mm²/30 min) | | | | |
| Exiting Cells (cells/mm²/30 min) | 7.0 ± 1.0 | 8.0 ± 1.5 | 8.3 ± 1.6 | 0.7 ± 0.6 |
| Motive Cells (%) | 10.0 | 30.5 | 90.0 | 96.0 |

### Example 4

Most previous animal model studies suggest a Th2 paradigm for allergic diseases, with increased activation of Th2 cells that produce Th2 cytokines resulting in the recruitment and activation of eosinophils. Eosinophils infiltrate into the lung 2 or 3 days after an allergen challenge and form inflammatory foci in the peribronchiolar and perivascular regions of the lung. We hypothesized that the focus areas of Th2 cells after OVA inhalation observed in the above experiments may coincide with those of eosinophils. To confirm this hypothesis, we investigated by immunohistochemistry of the lung after an allergen challenge.

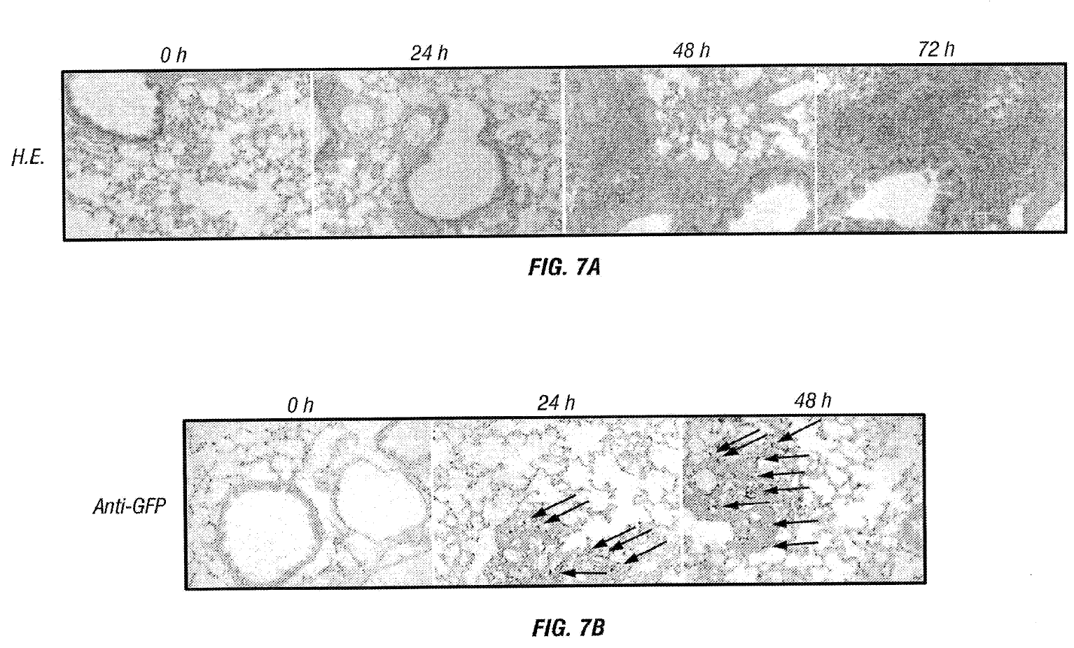

GFP⁺ OT2-Th2 cells were intravenously transferred into C57BL/6 mice. Two days later, recipient mice were exposed to an allergen challenge by OVA inhalation. Infiltration and focus formation of eosinophils were observed by H&E staining (FIG. 7a). Infiltrated OT2-Th2 cells were detected by immunohistochemistry with an anti-GFP antibody (FIG. 7b). Twenty-four hours after OVA inhalation, GFP⁺ OT2-Th2 cells infiltrated into the lung and formed foci, but eosinophils did not infiltrate (FIG. 7). Forty-eight hours after OVA inhalation, inflammatory cell infiltration into the lung was observed, and focus areas of inflammatory cells and GFP⁺OT2-Th2 cells coincided. Infiltration and foci formation of eosinophils remained at 72 hours after OVA inhalation. These results indicate that OT2-Th2 cells infiltrate into the lung in advance of eosinophils after allergen exposure, and might regulate the formation of inflammatory foci.

1. A laboratory rodent model for asthma wherein said rodent has been provided allergen-sensitized, fluorescently labeled lymphocytes which can be detected after inducing an asthmatic response to said allergen.

2. The model of claim 1, wherein said fluorescently labeled lymphocytes are harvested from a donor animal sensitized to the allergen.

3. The model of claim 1, wherein said fluorescently labeled lymphocytes are Th2 cells sensitized with the allergen in vitro.

4. The model of claim 2, wherein said allergen is selected from a group consisting of OVA, OVA peptide, sheep red blood cells, dsRNA, cockroach (rBla g2), house dust mite (rDer f1), house dust mite-extract, olive pollen (natural and recombinant Ole e1), Aspergillus fumigatus-extract, Timothy grass pollen (rPhl p5), birch pollen (rBet v1), rye grass pollen (Lol p1), olive pollen extract, Alternaria alternate-extract, Cladosporium herbarum-spores, Dermatophagoides pteronyssinus-extract, heat-coagulated hen's egg white, etc., or any combination thereof.

5. The model of claim 3, wherein said allergen is selected from a group consisting of OVA, OVA peptide, sheep red blood cells, dsRNA, cockroach (rBla g2), house dust mite (rDer f1), house dust mite-extract, olive pollen (natural and recombinant Ole e1), Aspergillus fumigatus-extract, Timothy grass pollen (rPhl p5), birch pollen (rBet v1), rye grass pollen (Lol p1), olive pollen extract, Alternaria alternate-extract, Cladosporium herbarum-spores, Dermatophagoides pteronyssinus-extract, heat-coagulated hen's egg white, etc., or any combination thereof.

6. The model of claim 2, wherein said allergen is administered by intraperitoneal, intranasal, intratracheal, or subcutaneous injection.

7. The model of claim 2, wherein said fluorescence is due to a transgene encoding a fluorescent protein.

8. The model of claim 1, wherein said lymphocytes are T lymphocytes.

9. The model of claim 8, wherein said lymphocytes are CD4⁺ T cells.

10. The model of claim 8, wherein said lymphocytes are Th2 cells.

11. The model of claim 8, wherein said lymphocytes are a mixture of different cell types.

12. A method of monitoring asthmatic responses, which method comprises:
a) administering said allergen to the rodent model of claim 1; and
b) detecting the presence, absence, or amount of the fluorescently labeled lymphocytes in the lungs of the rodent.

13. The method of claim 12, wherein said administering is by inhalation, intraperitoneal, intranasal, intratracheal, or subcutaneous injection.

14. The method of claim 12, wherein said fluorescently labeled lymphocytes are detected by whole-body optical imaging.

15. A method to determine the effectiveness of a candidate anti-asthma substance, which method comprises:
a) administering the allergen and a candidate anti-asthma substance to the rodent model of claim 1;
b) detecting the amount of lymphocytes in the lungs of the rodent model; and
c) comparing the amount determined in b) with the amount in a control rodent model not administered the substance,
wherein a decrease in the amount in the model in b) as compared to the control model indicates an anti-asthma effect of the substance.

16. The method of claim 15, wherein the administering of the allergen is by inhalation, intraperitoneal, intranasal, intratracheal, or subcutaneous injection.

17. The method of claim 15, wherein said fluorescently labeled lymphocytes are detected by whole-body optical imaging.
